(19) United States
(12) Patent Application Publication
(10) Pub. No.: US 2026/0043093 A1
(43) Pub. Date: Feb. 12, 2026

(54) CO-DOMINANT MOLECULAR MARKERS FOR LONG-DAY ADAPTATION GENE StCDF1.4 IN POTATO AND USE THEREOF

(71) Applicants: AGRICULTURAL GENOMICS INSTITUTE, CHINESE ACADEMY OF AGRICULTURAL SCIENCES, Shenzhen, Guangdong (CN); AGRICULTURAL GENOMICS INSTITUTE AT SHENZHEN, CHINESE ACADEMY OF AGRICULTURAL SCIENCES (SHENZHEN BRAN, Shenzhen (CN)

(72) Inventors: Chunzhi ZHANG, Shenzhen (CN); Lianlian MA, Shenzhen (CN); Zefeng ZHAI, Shenzhen (CN); Haicai LI, Shenzhen (CN)

(21) Appl. No.: 19/366,486

(22) Filed: Oct. 22, 2025

Related U.S. Application Data
(63) Continuation-in-part of application No. PCT/CN2025/094609, filed on May 13, 2025.

(30) Foreign Application Priority Data
May 13, 2024 (CN) 202410589473.3

(51) Int. Cl. C12Q 1/6895

(52) U.S. Cl. CPC C12Q 1/6895; C12Q 2600/13; C12Q 2600/16

(57) ABSTRACT

Co-dominant molecular markers for long-day adaptation gene StCDF1.4 in potato and the use thereof, which belongs to the technical field of molecular biology. The co-dominant molecular marker alone may identify homozygous or heterozygous genotypes of StCDF1.4, thus overcoming the limitation of existing molecular markers that require combinations of multiple pairs of primers to distinguish genotypes of StCDF1.4.

### CROSS-REFERENCE TO RELATED APPLICATION

The application is a continuation-in-part of International Patent Application No. PCT/CN2025/094609, filed on May 13, 2025, which claims priority of the Chinese patent application with the application number “202410589473.3”, filed on May 13, 2024, both of which are incorporated by references in their entities.

### REFERENCE TO SEQUENCE LISTING

A computer readable XML file entitled “SEQUENCE LISTING”, that was created on Sep. 1, 2025, with a file size of about 26849 bytes, contains the sequence listing for this application, has been filed with this application, and is hereby incorporated by reference in its entirety.

### TECHNICAL FIELD

The present disclosure relates to co-dominant molecular markers for the potato long-day adaptation gene StCDF1.4 in potato and the use thereof, which belongs to the technical field of molecular biology.

### BACKGROUND

Seasonal fluctuations in day length are the most important environmental factor affecting potato tuberization. Potatoes (Solanum tuberosum) originated in low-latitude regions near the equator. Wild-types potatoes are extremely sensitive to photoperiod, forming tubers under strictly short-day conditions but failing to form tubers under long-day conditions. StCDF1 is a key gene that regulates potato tuberization under long-day conditions and growth period. Four StCDF1 alleles have been identified in potatoes, namely StCDF1.1, StCDF1.2, StCDF1.3, and StCDF1.4. Among them, the StCDF1.4 genotype is the most widely distributed in potato cultivars adapted to long-day conditions, suggesting that StCDF1.4 has extremely high breeding value.

However, due to the high similarity among the alleles StCDF1.1, StCDF1.2, StCDF1.3 and StCDF1.4, especially the fact that StCDF1.2 and StCDF1.4 have the same number of base insertions (both with 7 base insertions) at the same position, people currently may only determine whether a potato variety contains the StCDF1.4 genotype through the combination of multiple primer pairs and tedious analyses. There has been a lack of effective molecular markers for identifying the StCDF1.4 genotype.

In addition, different StCDF1 genotypes exhibit distinct latitude-dependent ecological adaptations and developmental phase variations. If novel natural variations may be exploited and novel StCDF1 genotypes discovered, the resource options for potato breeding will be enriched, thereby enhancing adaptability to rapidly changing climatic conditions.

### SUMMARY

In view of the disadvantages of existing molecular markers, the present disclosure provides co-dominant molecular markers for long-day adaptation and growth period gene StCDF1.4 in potato, which enable rapid identification and screening for potato germplasm carrying the StCDF1.4 allele with only a single pair of primers. The identification work is time-efficient, labor-saving and environmentally independent, and delivers accurate and reliable results.

In addition, the present disclosure further provides a novel gene StCDF1.6. The mutant genotype is discovered in the cultivated potato variety E'malingshu 5 as a natural variant. Specifically:

Firstly, the present disclosure provides a promoter sequence of StCDF1.4, which enables the development of a pair of efficient and stable co-dominant molecular markers that effectively distinguish StCDF1.4 from other StCDF1 alleles. The molecular markers may overcome the limitation of existing molecular markers that require combinations of multiple pairs of primers to distinguish genotypes of StCDF1.4.

(1) The present disclosure provides for the first time a promoter sequence of StCDF1.4, and discovers for the first time that the promoter of StCDF1.4 has a 623 bp deletion compared to those of StCDF1.1, StCDF1.2 and StCDF1.3.

(2) The present disclosure develops a pair of efficient and stable co-dominant molecular markers capable of distinguishing StCDF1.4 from other StCDF1 alleles (with marker primers CDF1.4-prom F/R). The molecular markers may identify homozygous or heterozygous genotypes of StCDF1.4, thus overcoming the limitation of existing molecular markers that require combinations of multiple pairs of primers to distinguish genotypes of StCDF1.4.

(3) The amplification products of the molecular markers provided in the present disclosure (with marker primers CDF1.4-prom F/R) show significant differences in the size of characteristic bands. Whether the potato to be tested contains the StCDF1.4 gene and genotype thereof may be judged by agarose gel electrophoresis. The identification features low cost, high efficiency, no need for restriction digestion, and simple operation, may shorten the breeding cycle for long-day adaptation and growth period traits in potato germplasm, and effectively reduce breeding costs.

(4) Assisted breeding using the molecular markers provided in the present disclosure (with marker primers CDF1.4-prom F/R), may achieve accurate and efficient detection of the StCDF1.4 genotype in progeny, as well as non-destructive detection of potato seedlings. The molecular markers are suitable for extensive screening of the StCDF1.4 genotype in commercial large-scale breeding and for identification of the StCDF1.4 allele in potato germplasm resources.

Secondly, the present disclosure provides a novel mutant allele of potato StCDF1, StCDF1.6, and a sequence thereof, which enables the development of a CAPS marker for identifying StCDF1.6.

(1) The present disclosure discovers for the first time a novel allele StCDF1.6 associated with potato long-day adaptation and growth period, enriching the polymorphism of StCDF1 genotypes adapted to different latitudes in the field.

(2) The present disclosure verifies for the first time the function of StCDF1.6 in promoting tuberization under long-day conditions through transgenic experiments, enriching the breeding options for long-day adaptation genes.

(3) The present disclosure develops a pair of CAPS molecular markers for detecting the StCDF1.6 genotype, featuring high accuracy and high specificity, which enables rapid screening of test materials and genotyping, and thus has broad application prospects in potato photoperiod breeding and molecular marker-assisted breeding.

Finally, the present disclosure provides a kit capable of simultaneously detecting StCDF1.4 and StCDF1.6, which greatly facilitates the breeding of long-day adapted potato germplasm and molecular marker-assisted breeding.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to illustrate the technical solutions in examples of the present disclosure or in the prior art more clearly, a brief introduction to the accompanying drawings required for the examples will be provided below. Obviously, the accompanying drawings in the following description merely illustrate some of the examples of the present disclosure, and those of ordinary skill in the art may also obtain other drawings according to these drawings without involving any inventive effort.

FIG. 1 is a diagram showing alignment of the promoter sequences of StCDF1.1 (SEQ ID NO: 9), StCDF1.2 (SEQ ID NO: 10), StCDF1.3 (SEQ ID NO: 11), StCDF1.4 (SEQ ID NO: 12) and the consensus sequence (SEQ ID NO: 13) in Example 1.

FIG. 2 is a diagram showing agarose gel electrophoresis detection of the PCR amplification products in Example 3.

FIG. 3 is a diagram showing the StCDF1.4 transgenic positive seedlings in Example 5.

FIG. 4 is a diagram showing alignment of the StCDF1 allele sequences (StCDF1.1: SEQ ID NO: 14; StCDF1.2: SEQ ID NO: 15; StCDF1.3: SEQ ID NO: 16; StCDF1.4: SEQ ID NO: 17; StCDF1.6: SEQ ID NO: 18; and Consensus: SEQ ID NO: 19) in Example 6.

FIG. 5 is a diagram showing the StCDF1.6 transgenic positive seedlings in Example 7.

FIG. 6 is a diagram showing yeast two-hybrid assay between StCDF1.6 and StFKF1, StGI in Example 8.

FIG. 7 is a diagram showing polyacrylamide gel electrophoresis detection of the digested products in Example 10.

### DETAILED DESCRIPTION OF THE EMBODIMENTS

The present disclosure provides a reagent or kit, including a formulation for detecting the presence or absence of a large-fragment deletion in a promoter of potato StCDF1.

In some implementations, the potato StCDF1 is StCDF1.1, StCDF1.2, StCDF1.3, StCDF1.4 and/or StCDF1.6.

In some embodiments, the formulation for detecting the presence or absence of a large-fragment deletion in a promoter of potato StCDF1 is primer pair A:

CDF1.4-prom F:
(SEQ ID NO: 5)
TGATATTATCCATTTTGCCT;
CDF1.4-prom R:
(SEQ ID NO: 6)
GTAGAAAGGTGGATTAGAAT.

In some embodiments, the reagent or kit further includes primer pair B for detecting StCDF1.6:

StCDF1.6-CAPS F:
(SEQ ID NO: 7)
TCACAATCACGGAGAAGAGA;
StCDF1.6-CAPS R:
(SEQ ID NO: 8)
CTAGTGTTGACCATATAGAG.

The present disclosure provides a promoter, where the promoter has the sequence of SEQ ID NO: 1, specifically:

attccatgatctttcttaccatatctggaagtacctctaactcatcctgagctagaagttatggcaatttgaagttggccaaaactcaaatttt
cctaacttgaacaaatttccagattttcattatttccaaaaatgactatttccagttcttaacttcttcctagctatttcaaattgcgagatgt
tacataaatctcactcatgaacttaaacaatgaacaaacactctcattgaatagatgagcttaaagggatatgaaggctagaaccatgttttca
aagacctaagtcaaccgcgagcccaaacctccctactttatcttcacttcatccttataaacttgttggactatcgatcatttcttccttttaa
attagatgaattatatcattcaagagcttctttagtccttttgattagttgcttcacaaaaaattgtaggtgtggcaaggtaacagtcatggtt
taagtggattgagtacatgatgtttttccaatcttatttcaacaattcaatccgctagtgatattgtctattttggatctaagttcgcacgacc
ttaaaacacgtcactagtatgtaaggtcaacttacttatatatacaatatctctcatctgttttgttaatgtaggacttcttatcctaaattgg
agtgtcacaaataccaattcttatggatttagcgtcctcgttgagatttgtcctaccacattgagtttgtctaaactcaacactgagatttgcc
ccatcctattgagattgtctaaactcagttgaactttgtctcacatcgacaatcacatcgagagtgactctataccatctgtaacaatccaatc
cgctaatgatattatccattttgcctaggttgtacaatattaaaacgtgtcactgatatgtaattaaaatatgttttctctaacagtttaagtt
tttagatgagtcgatcacacacttcaaacgtaatgacttttgatagagaatccattttcataactattctaatccacctttctacaacattctt
atccatgatagtaaaaaaaaattaaaacagaaatagatattaaggcaaatctttattttctctttagtattaggccaaatctatttgagaaaaa
aattgggaaagtagttgctgacatggacaaataagaaagggagaaaatagtaccaaaagcacttgcctttttacttttagttttgggtacagaa
tcaaaaaaacagcaaacaaaccacacacacctaaaaagaacaaaaccaactctcccctccctcaaaccaaatgtatcaagatttctctattgcc
acatacaaatttgtagctacaaatccctctacctttagtgacgtgtccaccccaccccccacctctaaaaccctttttcttcttcatctatagt
cctcatagtccaaaacttataagacaccaaacatcaaagaaacaaaacaagaagataaaaatttgttcccatcttaccctttttcaagtaacac
aaactactttgatttctttccccccttttgagttgtaaaaaaaaagaaggaaaatcaagaattgagaagaggaattcaagaaa.

The present disclosure provides a molecular marker primer pair, where sequences of the primer pair are:

CDF1.4-prom F:
(SEQ ID NO: 5)
TGATATTATCCATTTTGCCT;
CDF1.4-prom R:
(SEQ ID NO: 6)
GTAGAAAGGTGGATTAGAAT.

The present disclosure provides the use of the above reagent or kit, promoter or molecular marker primer pair in identification of potato StCDF1.4.

In some embodiments, the identification of potato StCDF1.4 includes determining whether the potato contains StCDF1.4 gene, or identifying a homozygous or heterozygous genotype of StCDF1.4.

The present disclosure provides the use of the above reagent or kit, promoter or molecular marker primer pair in potato assisted breeding or variety improvement.

In some embodiments, potato assisted breeding or variety improvement includes marker-assisted breeding for long-day adaptation or early-, middle-, and late-maturing varieties of potato; screening long-day adaptation or early-, middle-, and late-maturing germplasm resources of potato; identifying long-day adaptation or growth period traits of potato.

The present disclosure provides a reagent or kit, including primer pair A for detecting StCDF1.4 and/or primer pair B for detecting StCDF1.6, where the primer pair A for detecting StCDF1.4 is:

CDF1.4-prom F:
(SEQ ID NO: 5)
TGATATTATCCATTTTGCCT;
CDF1.4-prom R:
(SEQ ID NO: 6)
GTAGAAAGGTGGATTAGAAT.

the primer pair B for detecting StCDF1.6 is:

StCDF1.6-CAPS F:
(SEQ ID NO: 7)
TCACAATCACGGAGAAGAGA;
StCDF1.6-CAPS R:
(SEQ ID NO: 8)
CTAGTGTTGACCATATAGAG.

The present disclosure provides a StCDF1.6 gene, where the StCDF1.6 gene has the nucleic acid sequence of SEQ ID NO: 2, specifically:

agcaaacaaaccacacacacctaaaaagaacaaaaccaactctcccctccctcaaaccaaatgtatcaagatttctctattgccacatacaaa
tttgtggctacaaatccctctacctttagtgacgtgtccaccccaccccccacttctaaaaccctttttcttcttcatctatagtcctcatag
tccaaaacttataagacaccaaacatcaaagaaacaaaacaagaagataaaaatttgttcccatcttaccctttttcaagtaacacaaactac
tttgatttctttccccctttttgtgttgtaaaaaaaagaaggaaaatcaagaattgagaagaggaattcaagaaaatgtctgaagttagagat
cctgctattaagctgtttggtaaaacaattggtatgacacaacaagaaaccaattgtgtttatcttcatgatgatcatacaacctcatcccct
ctttcaattgatgatgataaggtaaatctttttaccaaattgtttataaatatatcatgagttaataaaaatggaatttgggtcaccaatttt
agttataaagtttgaaactttataattgatatatctatttgaccataaaatttgatcaaacttttaaaaatatgatctttttaggacttgtct
cattttttttcaagtaaaaattcaaattttccagtttcaacttcaaaatctacgactagacggagttttagtatgtgggtgagttttgtttta
tgaaaagattggttctttcttgataccatagcttaaaatggaaatgtaaaaaaaaacagttttaggaaagaccctgttttgggaattgttttc
tcattatttttctgtatttgaatttggaaatttactgttttgggattatattttattttattttttcaatttagataccatagaactctgatg
tttgatctggtgatgttaatcaacatttaaaatatatatatatatatatggatatgatcttactttccaagagattttttttatatgtttctg
gagttaagtatgttaaagttgtgtaatttggaaagcacaaggattatatgccctgaaatattcagatcctgagggtgtagcggaaacggtctc
tctatcctaaaaagatagggtaacgtctacgtacatcctacactcttcagatcccatttgtgggattacaccgggtatgttgttgttattgta
gttgtatatattcagatcctctgcgttgtaaaacttttgtatttatggtgatcatattgttatgtgttttatcaagtggaattgttaacaact
gctaggagtggagatttaggcgtaaatgcccggatcgtttaccttgttcaattgcttatgctaaaatgatgagatcgtcgtcttttcagtgac
gaatagttttgatttgttgtgagagagctcaaactgaccttttttttcttgtcaattgatcagatcactttagaaggagaatttacacaaagc
aaacaagatgatgaacttgtcgatccaactgcagactcgtcgattgaaccagaaacatcatctggtataagcgatgacatcaagatgcaggat
gcagataaagaaacattatcatcaaaatctatcgaggaggaggattcgagtgaggaaaaggcactcaagaagcctgataaattgattccatgt
ccgcgatgtaatagcatggaaacaaagttctgttattacaacaattacaacgtcaaccagcctcgttacttctgcaagaactgccagagatat
tggactgctggagggacaatgagaaatgtgcctgtgggatctggtcgccgaaagaacaagagttcttccatttcaaattatcctcttcaagca
ggtcgggtcgaagcagcagctcacggaatgcatcttcctgctttaaggacaaatggaactgtccttacatttggatcagataaacccctttgt
gattcaatggtttctgcattgaacttagctgagaattcacataatatgaatcgaaatgaattccatggatccgaacgaagaatgcctgcaatc
gggaatgatcaatcaaatggaacttgtagtacagcctcaagtgtaactgacaaagaaagcagtgctggtactcatgatttagcaaattggaat
aatttccagccatttcctcctcaagtaccgtactttcagggcgctccgtggccttattctggctttccagtatcattctatccagcaacaccg
tactggggctgcaccgtagcaaacccttggaacgtaccttggctttcttctgatcaatcgtcagtccagaacaacagtcctacttcaccaaca
ttaggaaaacattctcgggatgaaagcaagattgatccatcacaatcacggagaagagatgctaatttgcagaatagagaaggcgagagatgt
gtactgattccgaagacattaaggattcatgatcaaatgaagcggctaaaagctctatatggtcaacactaggtatcaggaatgagaagattg
attcagctcgtggtacaatgctcttcagtgccttcaatccaaaagctgatcatagaaatcgcgaacatgacacttcttttgccctgcaagcta
atccagcagccttgtctagatcacttcatttccgcgagagcacacaatgataagacttacaacatggttcatagttgcctagtaccaatcatt
gatgttagtgcagctaacacctattcatggaaatagtctcttgcagaaatgcagggtaaagctgcgtacaatagacctttgtggactggccct
tcctcggacctgcgcatagcgggttcttgtgtctaggaggagaaggatacagtatgcacagagtcttcaagttttatagttgtgcagtactat
gtgtaccttgtacattgacctgtagagttattaatagatagagataaataagatgcagctgaagtttttactgacttctca.

The present disclosure provides a StCDF1.6 protein, where the StCDF1.6 protein has the amino acid sequence of SEQ ID NO: 4, specifically:

MSEVRDPAIKLFGKTIGMTQQETNCVYLHDDHTTSSPLSIDDDKITLEG
EFTQSKQDDELVDPTADSSIEPETSSGISDDIKMQDADKETLSSKSIEE
EDSSEEKALKKPDKLIPCPRCNSMETKFCYYNNYNVNQPRYFCKNCQRY
WTAGGTMRNVPVGSGRRKNKSSSISNYPLQAGRVEAAAHGMHLPALRTN
GTVLTFGSDKPLCDSMVSALNLAENSHNMNRNEFHGSERRMPAIGNDQS
NGTCSTASSVTDKESSAGTHDLANWNNFQPFPPQVPYFQGAPWPYSGFP
VSFYPATPYWGCTVANPWNVPWLSSDQSSVQNNSPTSPTLGKHSRDESK
IDPSQSRRRDANLQNREGERCVLIPKTLRIHDQMKRLKALYGQH.

The present disclosure provides a coding sequence for the StCDF1.6 protein described in the above solution. In some embodiments, the sequence is as set forth in SEQ ID NO: 3, specifically:

atgtctgaagttagagatcctgctattaagctgtttggtaaaacaattggtatgacacaacaagaaaccaattgtgtttatcttcatgatgatca
tacaacctcatcccctctttcaattgatgatgataagatcactttagaaggagaatttacacaaagcaaacaagatgatgaacttgtcgatccaa
ctgcagactcgtcgattgaaccagaaacatcatctggtataagcgatgacatcaagatgcaggatgcagataaagaaacattatcatcaaaatct
atcgaggaggaggattcgagtgaggaaaaggcactcaagaagcctgataaattgattccatgtccgcgatgtaatagcatggaaacaaagt
tctgttattacaacaattacaacgtcaaccagcctcgttacttctgcaagaactgccagagatattggactgctggagggacaatgagaaatgt
gcctgtgggatctggtcgccgaaagaacaagagttcttccatttcaaattatcctcttcaagcaggtcgggtcgaagcagcagctcacggaat
gcatcttcctgctttaaggacaaatggaactgtccttacatttggatcagataaacccctttgtgattcaatggtttctgcattgaacttagctg
agaattcacataatatgaatcgaaatgaattccatggatccgaacgaagaatgcctgcaatcgggaatgatcaatcaaatggaacttgtagtaca
gcctcaagtgtaactgacaaagaaagcagtgctggtactcatgatttagcaaattggaataatttccagccatttcctcctcaagtaccgtactt
tcagggcgctccgtggccttattctggctttccagtatcattctatccagcaacaccgtactggggctgcaccgtagcaaacccttggaacgta
ccttggctttcttctgatcaatcgtcagtccagaacaacagtcctacttcaccaacattaggaaaacattctcgggatgaaagcaagattgatcc
atcacaatcacggagaagagatgctaatttgcagaatagagaaggcgagagatgtgtactgattccgaagacattaaggattcatgatcaaa
tgaagcggctaaaagctctatatggtcaacactag.

The present disclosure provides a molecular marker primer pair, where sequences of the primer pair are:

StCDF1.6-CAPS F:
(SEQ ID NO: 7)
TCACAATCACGGAGAAGAGA;
StCDF1.6-CAPS R:
(SEQ ID NO: 8)
CTAGTGTTGACCATATAGAG.

The present disclosure provides the use of the above primer pair in identification of the StCDF1.6 gene or StCDF1.6 protein.

The present disclosure provides the use of the above primer pair in screening or identification of potato germplasm containing StCDF1.6.

The present disclosure provides the use of the above primer pair in potato assisted breeding or variety improvement.

In some embodiments, the potato assisted breeding is marker-assisted breeding for long-day adaptation or early-, middle-, and late-maturing varieties of potato.

The present disclosure provides a method for producing potato with long-day adaptation and growth period, the method including overexpressing the StCDF1.6 protein.

### EXAMPLES

The present disclosure is further elucidated by the following examples, but any examples or combinations thereof shall not be construed as limiting the scope or manner of implementation of the present disclosure. The experimental methods used in the examples below are all conventional methods unless otherwise specified. The materials, reagents, etc., used in the following examples are all commercially available, unless otherwise specified.

### Example 1: Cloning of Potato StCDF1.4 Gene and Development of Co-Dominant Molecular Markers Located in the Promoter Region

The genome resequencing (100×) was performed on 18 potato cultivars with relatively large cultivation areas in China, and StCDF1 alleles and copy numbers thereof in these cultivars were identified. The results are as shown in Table 1. The results show that of the 18 potato cultivars identified, the mutant allele StCDF1.4 is present in 14 cultivars, accounting for 77.8%; StCDF1.3 is present in eight cultivars, accounting for 44.4%; StCDF1.2 is present in two cultivars, accounting for 11.1%. The growth periods of 18 cultivars were collected. It is found that StCDF1.3 is generally present in early-maturing cultivars, and StCDF1.4 is present in both early-maturing and late-maturing cultivars (see Table 1).

TABLE 1
Identification of StCDF1 alleles in potato
cultivars with top promotion areas in China

| | Promotion | | Growth |
|---|---|---|---|
| Cultivar | area in 2020 | | period |
| name | (unit: 10,000 mu) | StCDF1 allele | (days) |
| Favorita | 824 | StCDF1.2/1.2/1.3/1.4 | 65 |
| Qingshu 9 | 699 | StCDF1.1/1.1/1.1/1.1 | 115 |
| Kexin 1 | 323 | StCDF1.1/1.1/1.3/1.4 | 90 |
| Jizhangshu 12 | 268 | StCDF1.1/1.1/1.2/1.4 | 96 |
| Mira | 253 | StCDF1.1/1.1/1.1/1.4 | 105 |
| Weiyu 3 | 168 | StCDF1.1/1.1/1.1/1.4 | 100 |
| Longshu 7 | 162 | StCDF1.1/1.1/1.1/1.4 | 115 |
| Weiyu 5 | 150 | StCDF1.1/1.1/1.1/1.4 | 95 |
| Atlantic | 115 | StCDF1.1/1.3/1.4/1.4 | 77 |
| Longshu 10 | 107 | StCDF1.1/1.1/1.1/1.4 | 110 |
| Zhongshu 5 | 101 | StCDF1.1/1.1/1.3/1.4 | 60 |
| Jinshu 16 | 100 | StCDF1.1/1.1/1.4/1.4 | 110 |
| Hezuo 88 | 97 | StCDF1.1/1.1/1.1/1.1 | 130 |
| Zaodabai | 89 | StCDF1.1/1.3/1.3/1.3 | 60 |
| Dongnong 303 | 75 | StCDF1.1/1.3/1.3/1.4 | 85 |
| Zhuangshu 5 | 72 | StCDF1.1/1.1/1.1/1.4 | 114 |
| Zhongshu 3 | 66 | StCDF1.1/1.3/1.3/1.4 | 60 |
| Xingjia 2 | 66 | StCDF1.1/1.3/1.3/1.3 | 83 |

Subsequently, the full-length gene sequence of StCDF1.4 (including promoter, gene exons, introns, and terminator) was cloned from the diploid potato germplasm C342 (StCDF1.1/1.4) for sequence analysis. The upstream primer sequence selected was located in a region 2 kb upstream from the start codon (ATG) and the downstream primer sequence was located in a region 855 bp downstream from the stop codon (TGA). The results show that in addition to a 7 bp insertion in the second exon (previously reported), StCDF1.4 has a large-fragment chromosomal replacement (629 bp->6 bp) in its promoter region at 672 bp upstream of the start codon, which has never been reported. The promoter sequence of StCDF1.4 is as set forth in SEQ ID NO: 1.

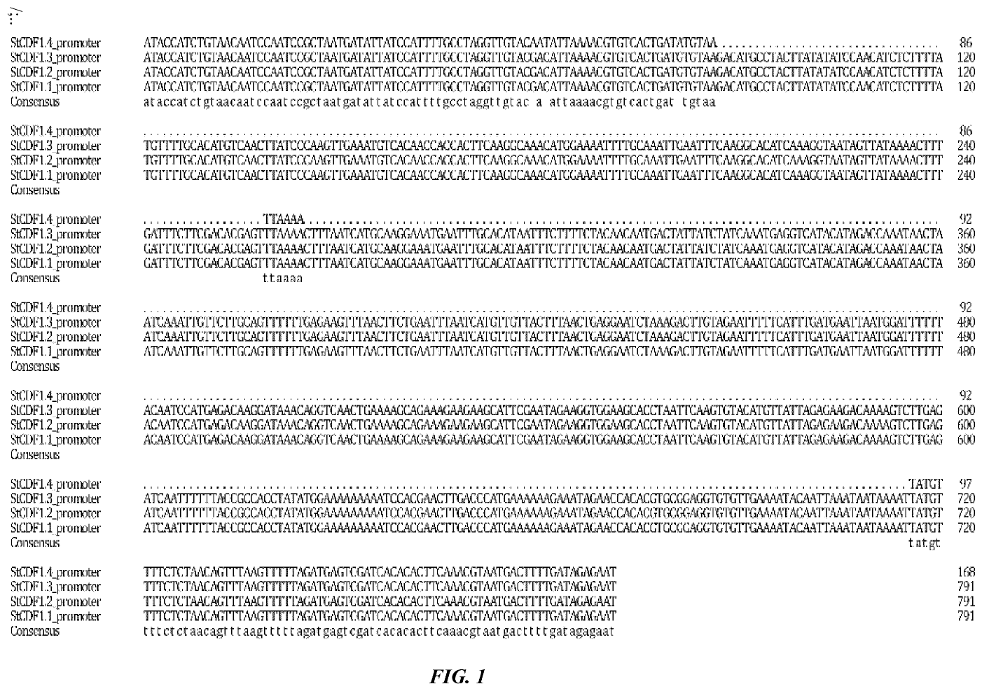

To determine whether this large-fragment chromosomal replacement in the promoter was specific to StCDF1.4, the inventors further cloned the promoter sequence of StCDF1.1 from the diploid potato germplasm C342 (StCDF1.1/1.4), and the promoter sequences of StCDF1.2 and StCDF1.3 from the diploid potato germplasms C894 (StCDF1.1/1.2) and C337 (StCDF1.1/1.3), respectively, and compared the promoter sequence of StCDF1.4 with the promoter sequences of StCDF1.1, StCDF1.2 and StCDF1.3. The results show that the presence of a large-fragment chromosomal replacement in the promoter region at a position 672 bp from the start codon is unique to StCDF1.4. This replacement results in a 623 bp deletion in the promoter of StCDF1.4 compared with those of StCDF1.1, StCDF1.2 and StCDF1.3 (as shown in FIG. 1).

The genomic sequence of potato had been published, and although many of the genes had been annotated, the start and end positions and functions of the full sequences or partial sequences of a large number of genes remained undetermined, such as the hypothetical proteins in genome annotations. With respect to the four alleles of StCDF1, it was currently known that: StCDF1.1 encodes a full-length protein, StCDF1.2 had a 7 bp insertion (CCACTAG) in the coding region of the second exon, StCDF1.4 also had a 7 bp insertion (GTATCCC) at the same position, and StCDF1.3 had an 865 bp transposon insertion. These variations resulted in frameshift mutations in StCDF1.2-1.4. Previous studies had mainly focused on the known mutations described above, yet never identified that StCDF1.4 exhibits a greater difference from StCDF1.1, StCDF1.2, and StCDF1.3 in the promoter region. Furthermore, this difference was not a variation or deletion of one or a few bases, but rather a large-fragment deletion. On the basis of the unexpected discovery, the inventors developed a pair of co-dominant molecular markers capable of distinguishing StCDF1.4 from other StCDF1 alleles. The primer sequences of the markers (CDF1.4-prom F/R) were as follows:

CDF1.4-prom F:
(SEQ ID NO: 5)
TGATATTATCCATTTTGCCT;
CDF1.4-prom R:
(SEQ ID NO: 6)
GTAGAAAGGTGGATTAGAAT.

### Example 2: Genotyping Method for Long-Day Adaptation and Growth Period Gene StCDF1.4 in Potato

(1) Genomic DNA was extracted from the potato to be tested.

(2) With the genomic DNA extracted in step (1) as a template, PCR amplification was performed on the template using the StCDF1.4 primer pair. The primer sequences were as follows:

CDF1.4-prom F:
(SEQ ID NO: 5)
TGATATTATCCATTTTGCCT;
CDF1.4-prom R:
(SEQ ID NO: 6)
GTAGAAAGGTGGATTAGAAT.

(3) PCR reaction system (10 μL): 5 μL of Green Mix, 0.5 μL of each of the two primers (10 μM), 1 μL of DNA template, and 3 μL of ddH₂O.

(4) PCR reaction conditions were as follows: pre-denaturing at 94-98° C. for 3-10 min; denaturing at 94-98° C. for 10-30 s, annealing at 52-60° C. for 10-30 s, and extension at 72° C. for 30-60 s, with a total of 25-35 cycles; extension at 72° C. for 3-10 min. Preferably, pre-denaturing at 95° C. for 3 min, denaturing at 95° C. for 30 s, annealing at 55° C. for 15 s, and extension at 72° C. for 30 s, with a total 30 cycles; extension at 72° C. for 5 min.

(5) The amplification products were analyzed using agarose gel electrophoresis. If the amplification product showed a characteristic band of 173 bp and no band of 796 bp, the potato to be tested was judged to be of a homozygous StCDF1.4 genotype; if the amplification product showed a characteristic band of 796 bp and no band of 173 bp, the potato to be tested was judged to be of a non-StCDF1.4 genotype; if the amplification product showed both 173 bp and 796 bp bands, the potato to be tested was judged to be of a heterozygous StCDF1.4 genotype.

### Example 3: Identification of StCDF1.4 Co-Dominant Molecular Markers in Potato Germplasm

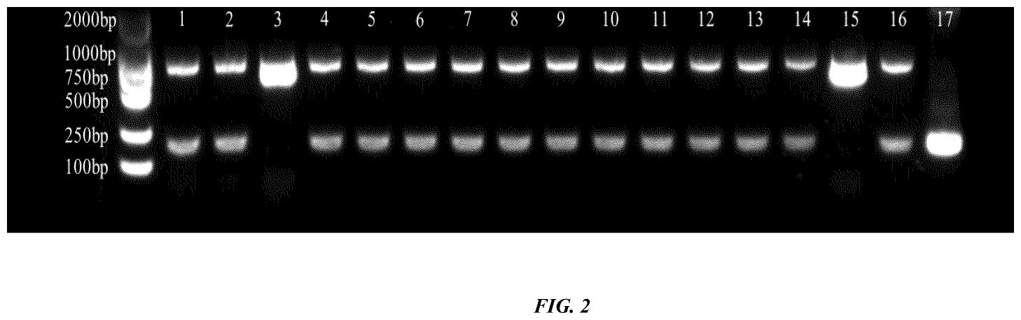

The StCDF1.4 co-dominant molecular marker (CDF1.4-prom F/R) was located in the StCDF1 promoter region on potato chromosome 5. When using this molecular marker for amplification, a characteristic band of 796 bp was produced for StCDF1.1, StCDF1.2 and StCDF1.3, while a characteristic band of 173 bp was obtained for StCDF1.4. To validate the prevalence of the 623 bp deletion of the StCDF1.4 promoter in the potato germplasm, 16 potato cultivars were selected here for the detection using the StCDF1.4 co-dominant molecular marker. Among them, 14 cultivars contained StCDF1.4, while two cultivars did not contain StCDF1.4 (see Table 2). The identification results are as shown in FIG. 2. All materials containing StCDF1.4 have the 173 bp characteristic band, materials without StCDF1.4 contain only the 796 bp characteristic band, and materials heterozygous for StCDF1.4 contain both 173 bp and 796 bp bands. It can be seen that the 623 bp deletion in the StCDF1.4 promoter is prevalent in various germplasm resources of potato; that is, the molecular marker or molecular marker primers of the present disclosure can be widely applied to the identification of potato StCDF1.4 genotypes.

TABLE 2
Cultivars used for validation of the
CDF1.4-prom F/R molecular marker

| No. | Cultivar name | StCDF1 allele |
|---|---|---|
| 1 | Favorita | StCDF1.2/1.2/1.3/1.4 |
| 2 | Kexin 1 | StCDF1.1/1.1/1.3/1.4 |
| 3 | Qingshu 9 | StCDF1.1/1.1/1.1/1.1 |
| 4 | Jizhangshu 12 | StCDF1.1/1.1/1.2/1.4 |
| 5 | Mira | StCDF1.1/1.1/1.1/1.4 |
| 6 | Weiyu 3 | StCDF1.1/1.1/1.1/1.4 |
| 7 | Longshu 7 | StCDF1.1/1.1/1.1/1.4 |
| 8 | Weiyu 5 | StCDF1.1/1.1/1.1/1.4 |
| 9 | Atlantic | StCDF1.1/1.3/1.4/1.4 |
| 10 | Longshu 10 | StCDF1.1/1.1/1.1/1.4 |
| 11 | Zhuangshu 5 | StCDF1.1/1.1/1.1/1.4 |
| 12 | Zhongshu 5 | StCDF1.1/1.1/1.3/1.4 |
| 13 | Jinshu 16 | StCDF1.1/1.1/1.4/1.4 |
| 14 | Zhongshu 3 | StCDF1.1/1.3/1.3/1.4 |
| 15 | Zaodabai | StCDF1.1/1.3/1.3/1.3 |
| 16 | Dongnong 303 | StCDF1.1/1.3/1.3/1.4 |
| 17 | Recombinant plasmid (containing | StCDF 1.4 |
| | full length of StCDF1.4 gene) | |

### Example 4: Use of StCDF1.4 Co-Dominant Molecular Markers in Potato Marker-Assisted Breeding and Variety Improvement

Potato molecular marker-assisted breeding and variety improvement were conventional methods in the field. The potato diploid D6 was a highly homozygous inbred line with advantages such as a long dormancy period and good fertility. However, potato diploid D6 failed to tuberize under long-day conditions (StCDF1.1/1.1), which limited its large-scale promotion in major northern production areas of China. By way of example only, to breed a variety having excellent traits such as long-day tuberization, long dormancy period and good fertility, the inventors selected C342 (StCDF1.1/1.4) as the male parent and the potato diploid D6 as the female parent for hybridization. The F1 generation and backcross populations were selected for StCDF1.4 genotypes using the StCDF1.4 co-dominant molecular marker. As a result, the breeding cycle and variety improvement of the D6 inbred line were accelerated.

### Example 5: Promotion of Tuberization Under Long-Day Conditions by StCDF1.4

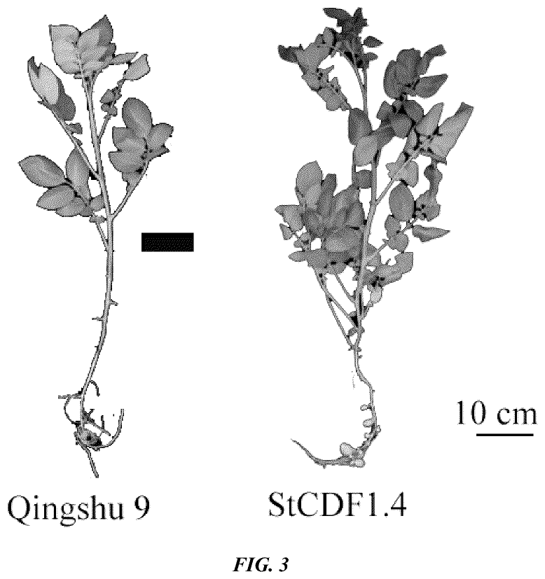

The full-length of the StCDF1.4 gene (including promoter, exons, introns, and terminator) was used to construct a recombinant plasmid with the PCAMBIA2300 vector. The recombinant plasmid was then introduced into the genetic background of Qingshu 9, where the expression of StCDF1.4 was driven by the promoter of StCDF1.4. A transgenic positive seedling and a control Qingshu 9 were grown under inductive long-day conditions (16 h light/8 h dark). After 36 days of growth under supplementary lighting conditions in the greenhouse, the StCDF1.4 transgenic positive seedling had obviously tuberized, while the control Qingshu 9 had not yet tuberized, as shown in FIG. 3. This indicates that StCDF1.4 can promote potato tuberization under long-day conditions.

### Example 6: Discovery of StCDF1.6 Allele

The commercial potato variety E'malingshu 5 was a mid-late maturing variety with a growth period of 94 days. Genomic DNA was extracted from the leaves of E'malingshu 5 for genome resequencing, with a sequencing depth of 100×. Three StCDF1 haplotypes were found in E'malingshu 5. Among them, haplotype 1 was StCDF1.1, which existed in two copies in E'malingshu 5; haplotype 2 was StCDF1.4 (with an identified insertion of 7 bases “GTATCCC”); haplotype 3 contained a C base deletion in the second exon. Since this haplotype had never been reported, the allele with the C base deletion was named as StCDF1.6.

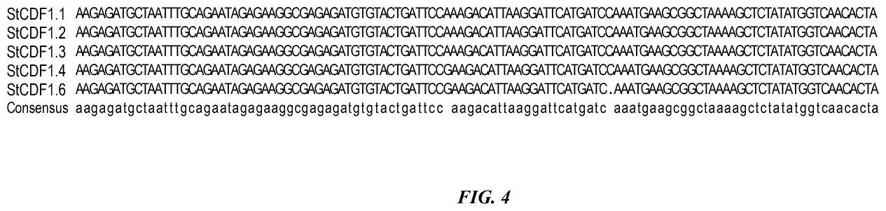

Due to a C base deletion in the coding region, StCDF1.6 underwent a frameshift mutation, resulting in a truncated protein that lacked the domain binding to StFKF1. Therefore, the inventors speculated that StCDF1.6 had the function of promoting tuberization under long-day conditions. The gene sequence of StCDF1.6 was as set forth in SEQ ID NO: 2, the CDS sequence was as set forth in SEQ ID NO: 3, and the amino acid sequence was as set forth in SEQ ID NO: 4. The diagram of nucleotide sequence alignment between StCDF1.6 and other StCDF1 alleles is as shown in FIG. 4.

### Example 7: Validation of Long-Day Adaptation Function of StCDF1.6

The full-length gene sequence of StCDF1.6 from E'malingshu 5, including promoter (2229 bp), exon, intron and terminator (1019 bp) was cloned and used to construct a recombinant plasmid with the pCAMBIA2300 vector. This recombinant plasmid was then introduced into the genetic background of Qingshu 9. After 39 days of growth of a transgenic positive seedling under artificial long-day conditions (16 h light/8 h dark), the StCDF1.6 transgenic positive seedling had tuberized, while Qingshu 9 had not yet tuberized, as shown in FIG. 5. This indicates that StCDF1.6 can promote tuberization under long-day conditions.

### Example 8: Loss of StFKF1-Binding Domain in StCDF1.6

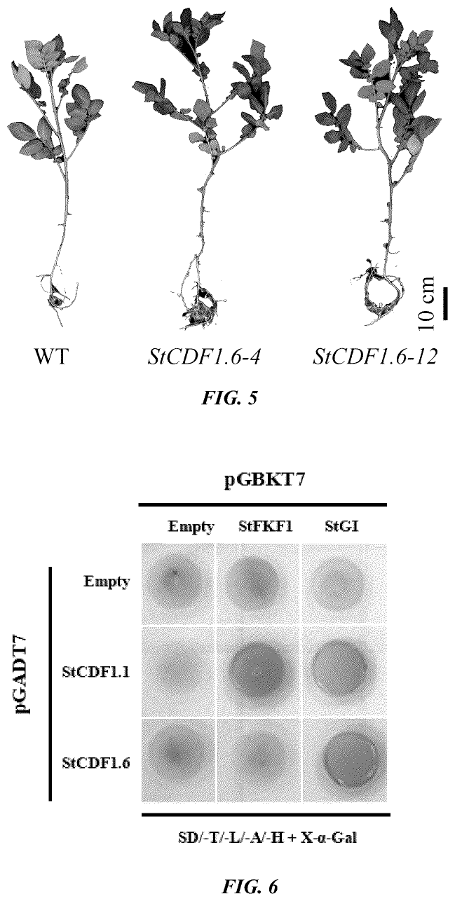

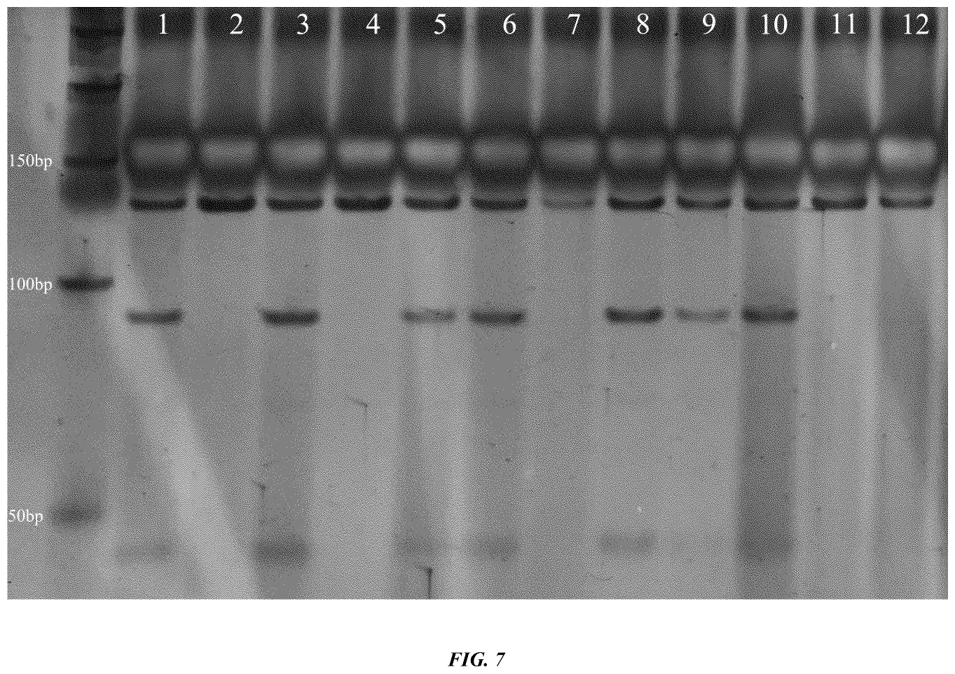

Due to a C base deletion, StCDF1.6 underwent a frameshift mutation, resulting in premature termination of the protein and loss of the StFKF1-binding domain. The inventors further speculated that StCDF1.6 cannot interact with StFKF1. To validate this, recombinant vectors were constructed by inserting StCDF1.1 and StCDF1.6 into pGADT7, and StFKF1 and StGI into pGBKT7, followed by transformation into yeast. The results show that StCDF1.6could not interact with StFKF1, but may interact with StGI (see FIG. 6).

### Example 9: Development of CAPS Molecular Marker Based on StCDF1.6 and Genotyping Method Therefor

The inventors found that the C base deletion in the coding region of StCDF1.6 occurred exactly at the Bcl I cleavage site, and thus developed a CAPS molecular marker. The primer sequences were shown as follows:

StCDF1.6-CAPS F:
(SEQ ID NO: 7)
TCACAATCACGGAGAAGAGA;
StCDF1.6-CAPS R:
(SEQ ID NO: 8)
CTAGTGTTGACCATATAGAG.

The genotyping method therefor was as follows:
(1) Genomic DNA was extracted from the potato to be tested.
(2) With the genomic DNA extracted in step (1) as a template, PCR amplification was performed on the template using the primers of the StCDF1.6 CAPS molecular marker (StCDF1.6-CAPS F/StCDF1.6-CAPS R).
(3) The amplification products were analyzed by agarose gel electrophoresis.
(4) Restriction digestion was performed on the amplification products using the Bcl I restriction endonuclease.
(5) The digested products were analyzed by polyacrylamide gel electrophoresis.

Preferably, the digested products were analyzed by polyacrylamide gel electrophoresis, and the genotypes were determined according to the types of bands of the digested products detected by electrophoresis.

The PCR reaction conditions for step (2) were as follows: pre-denaturing at 94-98° C. for 3-10 min; denaturing at 94-98° C. for 10-30 s, annealing at 52-60° C. for 10-30 s, and extension at 72° C. for 30-60 s, with a total of 25-35 cycles; extension at 72° C. for 3-10 min. Preferably, pre-denaturing at 95° C. for 3 min, denaturing at 95° C. for 30 s, annealing at 55° C. for 15 s, and extension at 72° C. for 30 s, with a total 30 cycles; extension at 72° C. for 5 min. PCR reaction system (10 μL): 5 μL of Green Mix, 0.5 μL of each of the two primers (10 μM), 1 μL of DNA template, and 3 μL of ddH₂O.

If bands of 127 bp and/or 126 bp appeared in step (3), the restriction digestion in step (4) could be performed.

The restriction digestion system (5 L) of step (4) was as follows: 2 μL of the PCR product of step (2), 0.5 μL of Bcl I, 1 μL of rCut Smart, and 1.5 μL of dd H₂O. The restriction digestion reaction was as follows: reaction at 37° C. for 30 min.

Step (5) was specifically as follows: If two bands of 84 bp and 42 bp appeared, the potato to be tested was judged to be of an StCDF1.6 genotype; if three bands of 127 bp, 84 bp and 42 bp appeared, the potato to be tested was judged to be of a heterozygous StCDF1.6 genotype; if a single band of 127 bp appeared, the potato to be tested was judged to not contain an StCDF1.6 genotype.

### Example 10: Use of CAPS Molecular Marker of StCDF1.6 in Breeding of Long-Day-Adapted Diploid Potato Germplasm

Haploid induction hybridization experiments of E'Malingshu 5 were performed using E'Malingshu 5 as the female parent and the haploid-inducing line PL4 with haploid induction ability as the male parent. Leaf DNA was extracted from the F1 generation. PCR amplification was performed using the CAPS molecular marker of StCDF1.6 (StCDF1.6-CAPS F/StCDF1.6-CAPS R). The amplified bands were subjected to restriction digestion with Bcl I restriction endonuclease, and the digested products were analyzed by polyacrylamide gel electrophoresis, as shown in FIG. 7. Lane 1 is E'Malingshu 5, with three bands of 127 bp, 84 bp and 42 bp, which is of the StCDF1.6 heterozygous genotype; Lane 2 is PL4, with only a 127 bp band, which does not contain the StCDF1.6 genotype; Lanes 3, 5, 6, 8, 9 and 10 are the F1 generation containing StCDF1.6, Lanes 4, 7, 11 and 12 are the F1 generation not containing StCDF1.6. The F1 generation containing the StCDF1.6 genotype was subjected to diploid identification, and diploid potato germplasm containing StCDF1.6 was selected.

The present disclosure has made every effort to describe the concept and the evidence for implementation effects of the invention. The scope of the present disclosure is defined by the appended claims. In view of the present specification and common general knowledge in the art, those of ordinary skill in the art may clearly understand the scope defined by the claims. Any modifications or changes in the technical solutions of the present disclosure may be made by those skilled in the art without departing from the spirit and scope of the present disclosure, and such modifications and changes are also included within the scope of the present disclosure.

What is claimed is:

1. A reagent or kit, comprising a formulation for detecting the presence or absence of a large-fragment deletion in a promoter of potato StCDF1.

2. The reagent or kit according to claim 1, wherein the potato StCDF1 is one or more of StCDF1.1, StCDF1.2, StCDF1.3, StCDF1.4, and StCDF1.6.

3. The reagent or kit according to claim 1, wherein the formulation for detecting the presence or absence of a large-fragment deletion in a promoter of potato StCDF1 is primer pair A, the primer pair A has the sequences of SEQ ID NO: 5 and SEQ ID NO: 6:
CDF1.4-prom F:
(SEQ ID NO: 5)
TGATATTATCCATTTTGCCT;
CDF1.4-prom R:
(SEQ ID NO: 6)
GTAGAAAGGTGGATTAGAAT.

4. The reagent or kit according to claim 3, further comprising primer pair B for detecting StCDF1.6, wherein the primer pair B has the sequences of SEQ ID NO: 7 and SEQ ID NO: 8:
StCDF1.6-CAPS F:
(SEQ ID NO: 7)
TCACAATCACGGAGAAGAGA;
StCDF1.6-CAPS R:
(SEQ ID NO: 8)
CTAGTGTTGACCATATAGAG.

5. A method for detecting potato StCDF1 genotype, comprising performing detection using the reagent or kit according to claim 1, wherein the identification of potato StCDF1 genotype comprises determining whether the potato contains StCDF1.4 gene, or identifying a homozygous or heterozygous genotype of StCDF1.4.

6. The method according to claim 5, wherein the identification of potato StCDF1 genotype further comprises determining whether the potato contains StCDF1.6 gene, or identifying a homozygous or heterozygous genotype of StCDF1.6.

7. The method according to claim 5, wherein comprising the following steps:
1) with the potato genomic DNA to be tested as a template, performing PCR amplification on the template using the primer pair A to obtain an amplification product; the primer pair A has the sequences of SEQ ID NO: 5 and SEQ ID NO: 6; and
2) analyzing the amplification product described in step 1) by agarose gel electrophoresis, wherein if the amplification product shows a characteristic band of 173 bp and no band of 796 bp, the potato to be tested is judged to be of a homozygous StCDF1.4 genotype; if the amplification product shows a characteristic band of 796 bp and no band of 173 bp, the potato to be tested is judged to be of a non-StCDF1.4 genotype; if the amplification product shows both 173 bp and 796 bp bands, the potato to be tested is judged to be of a heterozygous StCDF1.4 genotype.

8. The method according to claim 7, wherein a reaction system for the PCR amplification, based on 10 μL, comprises the following components: 5 μL of Green Mix, 0.5 μL of each of the two primers, 1 μL of DNA template, and 3 μL of ddH₂O; a concentration of each of the two primers is 10 μM.

9. The method according to claim 7, wherein reaction conditions for the PCR are: pre-denaturing at 94-98° C. for 3-10 min; denaturing at 94-98° C. for 10-30 s, annealing at 52-60° C. for 10-30 s, and extension at 72° C. for 30-60 s, with a total of 25-35 cycles; extension at 72° C. for 3-10 min.

10. The method according to claim 6, comprising the following steps:
(1) with the potato genomic DNA to be tested as a template, performing PCR amplification on the template using the pair B to obtain an amplification product, the primer pair B has the sequences of SEQ ID NO: 7 and SEQ ID NO: 8;
(2) performing restriction digestion on the amplification product using Bcl I restriction enzyme to obtain a digested product; and
(3) analyzing the digested product by polyacrylamide gel electrophoresis, wherein if two bands of 84 bp and 42 bp appear, the potato to be tested is judged to be of an StCDF1.6 genotype; if three bands of 127 bp, 84 bp and 42 bp appear, the potato to be tested is judged to be of a heterozygous StCDF1.6 genotype; if a single band of 127 bp appears, the potato to be tested is judged to not contain an StCDF1.6 genotype.

11. The reagent or kit according to claim 1, wherein the promoter of potato StCDF1 with a large-fragment deletion has the nucleotide sequence of SEQ ID NO: 1.

12. The reagent or kit according to claim 2, the StCDF1.6 encodes a protein having the amino acid sequence of SEQ ID NO: 4; and/or
the StCDF1.6 gene has the sequence of SEQ ID NO: 2; and/or
the StCDF1.6 gene has the coding region sequence of SEQ ID NO: 3.

13. A method for detecting potato StCDF1 genotype, comprising performing detection using the reagent or kit according to claim 11, wherein the identification of potato StCDF1 genotype comprises determining whether the potato contains StCDF1.4 gene, or identifying a homozygous or heterozygous genotype of StCDF1.4.

14. A method for detecting potato StCDF1 genotype, comprising performing detection using the reagent or kit according to claim 2, wherein the identification of potato StCDF1 genotype comprises determining whether the potato contains StCDF1.4 gene, or identifying a homozygous or heterozygous genotype of StCDF1.4.

15. A method for detecting potato StCDF1 genotype, comprising performing detection using the reagent or kit according to claim 12, wherein the identification of potato StCDF1 genotype comprises determining whether the potato contains StCDF1.4 gene, or identifying a homozygous or heterozygous genotype of StCDF1.4.

16. A method for detecting potato StCDF1 genotype, comprising performing detection using the reagent or kit according to claim 3, wherein the identification of potato StCDF1 genotype comprises determining whether the potato contains StCDF1.4 gene, or identifying a homozygous or heterozygous genotype of StCDF1.4.

17. A method for detecting potato StCDF1 genotype, comprising performing detection using the reagent or kit according to claim 4, wherein the identification of potato StCDF1 genotype comprises determining whether the potato contains StCDF1.4 gene, or identifying a homozygous or heterozygous genotype of StCDF1.4.


SEQUENCE LISTING (19 sequences)

SEQ ID NO: 1 - DNA, 1587 bp, Solanum tuberosum
  source 1..1587: mol_type = genomic DNA
attccatgatctttcttaccatatctggaagtacctctaactcatcctgagctagaagttatggcaatttgaagttggccaaaactcaaattttcctaacttgaacaaatttccagattttcattatttccaaaaatgactatttccagttcttaacttcttcctagctatttcaaattgcgagatgttacataaatctcactcatgaacttaaacaatgaacaaacactctcattgaatagatgagcttaaagggatatgaaggctagaaccatgttttcaaagacctaagtcaaccgcgagcccaaacctccctactttatcttcacttcatccttataaacttgttggactatcgatcatttcttccttttaaattagatgaattatatcattcaagagcttctttagtccttttgattagttgcttcacaaaaaattgtaggtgtggcaaggtaacagtcatggtttaagtggattgagtacatgatgtttttccaatcttatttcaacaattcaatccgctagtgatattgtctattttggatctaagttcgcacgaccttaaaacacgtcactagtatgtaaggtcaacttacttatatatacaatatctctcatctgttttgttaatgtaggacttcttatcctaaattggagtgtcacaaataccaattcttatggatttagcgtcctcgttgagatttgtcctaccacattgagtttgtctaaactcaacactgagatttgccccatcctattgagattgtctaaactcagttgaactttgtctcacatcgacaatcacatcgagagtgactctataccatctgtaacaatccaatccgctaatgatattatccattttgcctaggttgtacaatattaaaacgtgtcactgatatgtaattaaaatatgttttctctaacagtttaagtttttagatgagtcgatcacacacttcaaacgtaatgacttttgatagagaatccattttcataactattctaatccacctttctacaacattcttatccatgatagtaaaaaaaaattaaaacagaaatagatattaaggcaaatctttattttctctttagtattaggccaaatctatttgagaaaaaaattgggaaagtagttgctgacatggacaaataagaaagggagaaaatagtaccaaaagcacttgcctttttacttttagttttgggtacagaatcaaaaaaacagcaaacaaaccacacacacctaaaaagaacaaaaccaactctcccctccctcaaaccaaatgtatcaagatttctctattgccacatacaaatttgtagctacaaatccctctacctttagtgacgtgtccaccccaccccccacctctaaaaccctttttcttcttcatctatagtcctcatagtccaaaacttataagacaccaaacatcaaagaaacaaaacaagaagataaaaatttgttcccatcttaccctttttcaagtaacacaaactactttgatttctttccccccttttgagttgtaaaaaaaaagaaggaaaatcaagaattgagaagaggaattcaagaaa

SEQ ID NO: 2 - DNA, 2964 bp, Solanum tuberosum
  source 1..2964: mol_type = genomic DNA
agcaaacaaaccacacacacctaaaaagaacaaaaccaactctcccctccctcaaaccaaatgtatcaagatttctctattgccacatacaaatttgtggctacaaatccctctacctttagtgacgtgtccaccccaccccccacttctaaaaccctttttcttcttcatctatagtcctcatagtccaaaacttataagacaccaaacatcaaagaaacaaaacaagaagataaaaatttgttcccatcttaccctttttcaagtaacacaaactactttgatttctttccccctttttgtgttgtaaaaaaaagaaggaaaatcaagaattgagaagaggaattcaagaaaatgtctgaagttagagatcctgctattaagctgtttggtaaaacaattggtatgacacaacaagaaaccaattgtgtttatcttcatgatgatcatacaacctcatcccctctttcaattgatgatgataaggtaaatctttttaccaaattgtttataaatatatcatgagttaataaaaatggaatttgggtcaccaattttagttataaagtttgaaactttataattgatatatctatttgaccataaaatttgatcaaacttttaaaaatatgatctttttaggacttgtctcattttttttcaagtaaaaattcaaattttccagtttcaacttcaaaatctacgactagacggagttttagtatgtgggtgagttttgttttatgaaaagattggttctttcttgataccatagcttaaaatggaaatgtaaaaaaaaacagttttaggaaagaccctgttttgggaattgttttctcattatttttctgtatttgaatttggaaatttactgttttgggattatattttattttattttttcaatttagataccatagaactctgatgtttgatctggtgatgttaatcaacatttaaaatatatatatatatatatggatatgatcttactttccaagagattttttttatatgtttctggagttaagtatgttaaagttgtgtaatttggaaagcacaaggattatatgccctgaaatattcagatcctgagggtgtagcggaaacggtctctctatcctaaaaagatagggtaacgtctacgtacatcctacactcttcagatcccatttgtgggattacaccgggtatgttgttgttattgtagttgtatatattcagatcctctgcgttgtaaaacttttgtatttatggtgatcatattgttatgtgttttatcaagtggaattgttaacaactgctaggagtggagatttaggcgtaaatgcccggatcgtttaccttgttcaattgcttatgctaaaatgatgagatcgtcgtcttttcagtgacgaatagttttgatttgttgtgagagagctcaaactgaccttttttttcttgtcaattgatcagatcactttagaaggagaatttacacaaagcaaacaagatgatgaacttgtcgatccaactgcagactcgtcgattgaaccagaaacatcatctggtataagcgatgacatcaagatgcaggatgcagataaagaaacattatcatcaaaatctatcgaggaggaggattcgagtgaggaaaaggcactcaagaagcctgataaattgattccatgtccgcgatgtaatagcatggaaacaaagttctgttattacaacaattacaacgtcaaccagcctcgttacttctgcaagaactgccagagatattggactgctggagggacaatgagaaatgtgcctgtgggatctggtcgccgaaagaacaagagttcttccatttcaaattatcctcttcaagcaggtcgggtcgaagcagcagctcacggaatgcatcttcctgctttaaggacaaatggaactgtccttacatttggatcagataaacccctttgtgattcaatggtttctgcattgaacttagctgagaattcacataatatgaatcgaaatgaattccatggatccgaacgaagaatgcctgcaatcgggaatgatcaatcaaatggaacttgtagtacagcctcaagtgtaactgacaaagaaagcagtgctggtactcatgatttagcaaattggaataatttccagccatttcctcctcaagtaccgtactttcagggcgctccgtggccttattctggctttccagtatcattctatccagcaacaccgtactggggctgcaccgtagcaaacccttggaacgtaccttggctttcttctgatcaatcgtcagtccagaacaacagtcctacttcaccaacattaggaaaacattctcgggatgaaagcaagattgatccatcacaatcacggagaagagatgctaatttgcagaatagagaaggcgagagatgtgtactgattccgaagacattaaggattcatgatcaaatgaagcggctaaaagctctatatggtcaacactaggtatcaggaatgagaagattgattcagctcgtggtacaatgctcttcagtgccttcaatccaaaagctgatcatagaaatcgcgaacatgacacttcttttgccctgcaagctaatccagcagccttgtctagatcacttcatttccgcgagagcacacaatgataagacttacaacatggttcatagttgcctagtaccaatcattgatgttagtgcagctaacacctattcatggaaatagtctcttgcagaaatgcagggtaaagctgcgtacaatagacctttgtggactggcccttcctcggacctgcgcatagcgggttcttgtgtctaggaggagaaggatacagtatgcacagagtcttcaagttttatagttgtgcagtactatgtgtaccttgtacattgacctgtagagttattaatagatagagataaataagatgcagctgaagtttttactgacttctca

SEQ ID NO: 3 - DNA, 1164 bp, Solanum tuberosum
  source 1..1164: mol_type = other DNA
atgtctgaagttagagatcctgctattaagctgtttggtaaaacaattggtatgacacaacaagaaaccaattgtgtttatcttcatgatgatcatacaacctcatcccctctttcaattgatgatgataagatcactttagaaggagaatttacacaaagcaaacaagatgatgaacttgtcgatccaactgcagactcgtcgattgaaccagaaacatcatctggtataagcgatgacatcaagatgcaggatgcagataaagaaacattatcatcaaaatctatcgaggaggaggattcgagtgaggaaaaggcactcaagaagcctgataaattgattccatgtccgcgatgtaatagcatggaaacaaagttctgttattacaacaattacaacgtcaaccagcctcgttacttctgcaagaactgccagagatattggactgctggagggacaatgagaaatgtgcctgtgggatctggtcgccgaaagaacaagagttcttccatttcaaattatcctcttcaagcaggtcgggtcgaagcagcagctcacggaatgcatcttcctgctttaaggacaaatggaactgtccttacatttggatcagataaacccctttgtgattcaatggtttctgcattgaacttagctgagaattcacataatatgaatcgaaatgaattccatggatccgaacgaagaatgcctgcaatcgggaatgatcaatcaaatggaacttgtagtacagcctcaagtgtaactgacaaagaaagcagtgctggtactcatgatttagcaaattggaataatttccagccatttcctcctcaagtaccgtactttcagggcgctccgtggccttattctggctttccagtatcattctatccagcaacaccgtactggggctgcaccgtagcaaacccttggaacgtaccttggctttcttctgatcaatcgtcagtccagaacaacagtcctacttcaccaacattaggaaaacattctcgggatgaaagcaagattgatccatcacaatcacggagaagagatgctaatttgcagaatagagaaggcgagagatgtgtactgattccgaagacattaaggattcatgatcaaatgaagcggctaaaagctctatatggtcaacactag

SEQ ID NO: 4 - protein, 387 aa, Solanum tuberosum
MSEVRDPAIKLFGKTIGMTQQETNCVYLHDDHTTSSPLSIDDDKITLEGEFTQSKQDDELVDPTADSSIEPETSSGISDDIKMQDADKETLSSKSIEEEDSSEEKALKKPDKLIPCPRCNSMETKFCYYNNYNVNQPRYFCKNCQRYWTAGGTMRNVPVGSGRRKNKSSSISNYPLQAGRVEAAAHGMHLPALRTNGTVLTFGSDKPLCDSMVSALNLAENSHNMNRNEFHGSERRMPAIGNDQSNGTCSTASSVTDKESSAGTHDLANWNNFQPFPPQVPYFQGAPWPYSGFPVSFYPATPYWGCTVANPWNVPWLSSDQSSVQNNSPTSPTLGKHSRDESKIDPSQSRRRDANLQNREGERCVLIPKTLRIHDQMKRLKALYGQH

SEQ ID NO: 5 - DNA, 20 bp, synthetic construct
  source 1..20: mol_type = other DNA
tgatattatccattttgcct

SEQ ID NO: 6 - DNA, 20 bp, synthetic construct
  source 1..20: mol_type = other DNA
gtagaaaggtggattagaat

SEQ ID NO: 7 - DNA, 20 bp, synthetic construct
  source 1..20: mol_type = other DNA
tcacaatcacggagaagaga

SEQ ID NO: 8 - DNA, 20 bp, synthetic construct
  source 1..20: mol_type = other DNA
ctagtgttgaccatatagag

SEQ ID NO: 9 - DNA, 791 bp, synthetic construct
  source 1..791: mol_type = other DNA
ataccatctgtaacaatccaatccgctaatgatattatccattttgcctaggttgtacgacattaaaacgtgtcactgatgtgtaagacatgcctacttatatatccaacatctcttttatgttttgcacatgtcaacttatcccaagttgaaatgtcacaaccaccacttcaaggcaaacatggaaaattttgcaaattgaatttcaaggcacatcaaaggtaatagttataaaactttgatttcttcgacacgagtttaaaactttaatcatgcaaggaaatgaatttgcacataatttcttttctacaacaatgactattatctatcaaatgaggtcatacatagaccaaataactaatcaaattgttcttgcagttttttgagaagtttaacttctgaatttaatcatgttgttactttaactgaggaatctaaagacttgtagaatttttcatttgatgaattaatggattttttacaatccatgagacaaggataaacaggtcaactgaaaagcagaaagaagaagcattcgaatagaaggtggaagcacctaattcaagtgtacatgttattagagaagacaaaagtcttgagatcaattttttaccgccacctatatggaaaaaaaaatccacgaacttgacccatgaaaaaagaaatagaaccacacgtgcggaggtgtgttgaaaatacaattaaataataaaattatgttttctctaacagtttaagtttttagatgagtcgatcacacacttcaaacgtaatgacttttgatagagaat

SEQ ID NO: 10 - DNA, 791 bp, synthetic construct; identical to SEQ ID NO: 9
  source 1..791: mol_type = other DNA

SEQ ID NO: 11 - DNA, 791 bp, synthetic construct; identical to SEQ ID NO: 9
  source 1..791: mol_type = other DNA

SEQ ID NO: 12 - DNA, 168 bp, synthetic construct
  source 1..168: mol_type = other DNA
ataccatctgtaacaatccaatccgctaatgatattatccattttgcctaggttgtacaatattaaaacgtgtcactgatatgtaattaaaatatgttttctctaacagtttaagtttttagatgagtcgatcacacacttcaaacgtaatgacttttgatagagaat

SEQ ID NO: 13 - DNA, 165 bp, synthetic construct
  source 1..165: mol_type = other DNA
ataccatctgtaacaatccaatccgctaatgatattatccattttgcctaggttgtacaattaaaacgtgtcactgattgtaattaaaatatgttttctctaacagtttaagtttttagatgagtcgatcacacacttcaaacgtaatgacttttgatagagaat

SEQ ID NO: 14 - DNA, 112 bp, synthetic construct
  source 1..112: mol_type = other DNA
aagagatgctaatttgcagaatagagaaggcgagagatgtgtactgattccaaagacattaaggattcatgatccaaatgaagcggctaaaagctctatatggtcaacacta

SEQ ID NO: 15 - DNA, 112 bp, synthetic construct; identical to SEQ ID NO: 14
  source 1..112: mol_type = other DNA

SEQ ID NO: 16 - DNA, 112 bp, synthetic construct; identical to SEQ ID NO: 14
  source 1..112: mol_type = other DNA

SEQ ID NO: 17 - DNA, 112 bp, synthetic construct
  source 1..112: mol_type = other DNA
aagagatgctaatttgcagaatagagaaggcgagagatgtgtactgattccgaagacattaaggattcatgatccaaatgaagcggctaaaagctctatatggtcaacacta

SEQ ID NO: 18 - DNA, 111 bp, synthetic construct
  source 1..111: mol_type = other DNA
aagagatgctaatttgcagaatagagaaggcgagagatgtgtactgattccgaagacattaaggattcatgatcaaatgaagcggctaaaagctctatatggtcaacacta

SEQ ID NO: 19 - DNA, 110 bp, synthetic construct
  source 1..110: mol_type = other DNA
aagagatgctaatttgcagaatagagaaggcgagagatgtgtactgattccaagacattaaggattcatgatcaaatgaagcggctaaaagctctatatggtcaacacta